(19) United States
(12) Patent Application Publication
(10) Pub. No.: US 2014/0335147 A1
(43) Pub. Date: Nov. 13, 2014

(54) REGENERATION AID FOR BONE DEFECTS

(71) Applicant: Antonis Alexakis, Oehningen (DE)

(72) Inventor: Antonis Alexakis, Oehningen (DE)

(21) Appl. No.: 14/362,349

(22) PCT Filed: Nov. 26, 2012
(86) PCT No.: PCT/EP2012/073622

§ 371 Date: Jun. 2, 2014

(30) Foreign Application Priority Data
Dec. 1, 2011 (DE) 10 2011 119 909.1

(51) Int. Cl. A61L 27/34; A61L 27/12; A61L 27/36; A61L 27/58; A61L 27/02

(52) U.S. Cl. CPC A61L 27/34; A61L 27/58; A61L 27/025; A61L 27/3608; A61L 27/365; A61L 27/12; A61L 2430/02
USPC 424/426; 424/423; 514/16.7

(57) ABSTRACT

The invention relates to a molded part for supporting bone regeneration, in particular the regeneration of a jawbone or jawbone section in a mammal, preferably a human, wherein the molded part is suitable for applying to the jawbone and has a coating having a composition comprising at least one collagen, a granular material, and hyaluronic acid or a hyaluronic acid derivative. The invention further relates to a granular material that can be used in the coating, to a method for producing the granular material, and to the use of the molded part.

The invention relates to a molded part for supporting bone regeneration.

A plurality of applications exists in medicine, in which it is desirable that the bone material of human or animal patients is self-regenerated. It is known that osteoblasts aim to grow into cavities. This knowledge is utilized, for example, in dentistry, whenever the jawbone is affected, and partially destroyed, by periodontitis.

It is known that the cavity required for the targeted growth of osteoblasts on the human or animal jawbone may be formed by means of a barrier or a molded part or a molded body, respectively. Some materials that are available as a barrier or for forming a barrier body in order to fill bone defects or in order to perform, in particular in dentistry, jaw reconstruction in terms of height and/or width are also known. One disadvantage is that some of these materials are resorbed very quickly such that the goal of filling or reconstruction may not be achieved before the barrier has been completely resorbed. Although other materials are resorbed very slowly, they do not allow any osteoblast growth in the higher layers, as there no longer is any culture medium left for osteoblast growth due to the long residence time of the barrier.

A molded part is known, for example, from DE 10 2005 060 761 Al. Although said molded part shows a resorption time that is appropriate for bone growth, it has the disadvantage that a sufficient nutrient supply is not ensured by means of the molded part alone. The area of the molded part also lacks an environment facilitating cell growth.

The problem addressed by the present invention is therefore that of overcoming the disadvantages of the prior art and providing a molded part facilitating osteoblast growth.

This problem is solved by a molded part according to claim 1 and a granular material according to claim 14.

According to the invention, a molded body is provided which serves to support bone regeneration, in particular regeneration of a jawbone or of a jawbone section in a mammal, preferably in a human. For this purpose, the molded part is suitable for a substantially tight application on a bone substrate and has, according to the invention, a coating having a composition comprising at least one collagen, a granular material, and hyaluronic acid or hyaluronic acid derivative.

The tightly sealed cavity, which is formed underneath the molded body or which is defined by the molded part is subdivided into smaller spaces by means of the coating comprising granular material, collagen, and hyaluronic acid. A blood clot forming in the cavity is substantially stabilized and therefore increases the chances of vessels being able to simultaneously grow in the entire cavity. Said vessels serve for nutrient supply of the osteoblasts such that an ossification or osteogenesis may occur, during the course of which new bone material is formed, and therefore, for example, a jawbone previously damaged due to periodontitis may be reconstructed.

For this purpose, the molded part, being completed resorbable by the body, is embodied in a permanently dimensionally stable manner and dimensioned such that the resorption process is not completed until sufficient ossification or osteogenesis has been achieved, i.e. once a supporting or protecting molded part is no longer required. Preferably, the molded part according to the invention can be produced as a mass product in various sizes. For this purpose, the molded part is formed such that it can be used directly on or at the bone without any modifications, with the exception of minor corrections. For this purpose, the molded part is provided in various sizes and adapted to various usage positions. It is considered advantageous if both the coating and the molded part are completely resorbed by the human or animal body.

Numerous advantages arise from the uniform coating of the molded part according to the invention, comprising collagen, granular material and/or hyaluronic acid. Thus, blood of the patient is absorbed such that body cells are available at any location within the cavity. Additionally, the cavity underneath the molded part is subdivided into smaller spaces by means of the coating, in particular due to the granular material, thus ensuring simultaneous growth of vessels in the entire cavity. The vessels are of critical importance to the permanent nutrient supply of the osteoblasts, since new bone material can only be formed under sufficient nutrient supply. A further advantage of the coating is that same enables the adherence of the molded part to the existing bone substrate, thus facilitating the handling of the molded part, i.e. the insertion and anchoring on or in the remaining bone material.

Contrary to known molded parts, the coating comprising granular material, collagen, and hyaluronic acid improves the formation and stability of the formed and desired blood clot. Contrary to the known molded part, which merely forms a cavity in which bone cannot always grow, the blood clot does not break down in the molded part according to the invention due to the cavity being subdivided by the coating.

The collagens or collagen types provided in the composition take on a function as an adhesive in order to be able to coat the molded part, or the inner face thereof, with granular material. Additionally, collagen also promotes the formation of an extracellular matrix, therefore facilitating the growth and accumulation of osteoblasts or the adherence of the blood clot in the cavity of the molded part. In turn, regeneration of the bone is substantially improved thereby. Collagen is available in multiple types. These are designated as collagen types 1 to 29. In the context of the present invention, the use of collagen type 1 and/or collagen type 3 is predominantly proposed. However, the invention should not be limited thereto, but equally comprises the remaining types not further mentioned, insofar as the use thereof in the context of the present invention is considered reasonable and feasible. The collagens used are usually of animal source and come from tendons, ligaments and/or the skin of mammals. Of course, the invention also comprises synthetically produced collagens or the use thereof. As already specified, the use of the collagen in the context of the present invention facilitates osteoblast growth. Subsequent to sufficient accumulation of such cells, same can also independently carry out the collagen type 1 synthesis and therefore supplement or replace the externally incorporated collagen.

The hyaluronic acid (or hyaluronic acid derivatives) likewise used in the context of the present invention has a favorable effect on the treatment of pathological changes of the periodontium and shows positive effects on fibroblasts, bone regeneration and wound healing. In the context of the present invention, hyaluronic acid (or the derivatives thereof) can be directly added to, or mixed with the composition according to the invention. In parallel, or alternatively, it is possible that a composition, consisting of granular material and collagen, is initially produced, and same is applied to the inner face of the molded part as a coating. Subsequent to the preparation of the molded part and during the insertion or application on a bone substrate, the addition of, or flushing of the application site with, a hyaluronic acid preparation is effected such that ultimately a composition according to the invention is produced.

The hyaluronic acid has various functions. The fundamental mode of action of the hyaluronic acid in the context of the present invention provides that three-dimensional mesh networks are created in an aqueous environment as a result of a spontaneous aggregation of the hyaluronic acid chains. Cellular and fibrous components can be embedded therein. In this manner, the formation of a bone structure is facilitated and promoted. Simultaneously, hyaluronic acid has a regulating function in the organization of the extracellular matrix and the components thereof. For this purpose, the hyaluronic acid network formed represents a prerequisite for mass transfer and simultaneously serves as a barrier against the penetration of foreign substances. By forming the networks and the condensation thereof, cells can be protected from decomposition processes and hydroxyl radicals. The shells of hyaluronic acid thus present serve as protection of various cell types from exterior, such as viral or bacterial, influences, therefore also promoting the survival probability of the osteoblasts.

Furthermore, a negatively charged hyaluronic acid has the capacity of binding enormous amounts of water and various plasma proteins via hydrogen bonds and the polar ends, and therefore functions as a type of “osmotic buffer” of the extracellular matrix. Hyaluronic acid is also advantageous in combating centers of chronic inflammation and has an anti-inflammatory potential. Hyaluronic acid also influences cellular growth factors and therefore has a positive influence on cellular growth processes and therefore supports tissue regeneration. These numerous advantages are utilized in the context of the present invention or in the composition serving as the coating. It was surprisingly found that regeneration of the bone or bone material can be significantly improved. Therefore, a clearly superior form of ossification or osteogenesis is effected as opposed to the prior art, which results from, among others, the composition according to the invention and the contained or released hyaluronic acid in combination with the remaining components.

The invention provides that a composition comprising or consisting of granular material, collagen, and hyaluronic acid is used for the coating. For this purpose, the collagen is in particular a mixture of collagen type 1 and collagen type 3. However, it is also conceivable to utilize only collagen type 1 or collagen type 3 exclusively. Collagen possesses not only sealing properties, but also fixes the molded body, at least temporarily, due to the adhesive effect thereof. The coating of granular material, collagen, and hyaluronic acid subdivides the cavity into multiple small spaces and thereby stabilizes the forming blood clot.

Preferably, the coating is provided on a surface of the molded part facing the bone substrate. The adhesive property of the at least one collagen is also utilized for the adhesion of the granular material on the molded part or the surface thereof, as it was already utilized in the above described adhesion of the molded part on the bone. As an alternative or additionally, a fibrin glue may be used here.

The composition of the coating preferably comprises:
1 to 10%, in particular 2 to 7.5%, preferably 5% of collagen,
99 to 80%, in particular 96 to 90%, preferably 95% of granular material, and
0.01 to 2%, in particular 0.5 to 1.5%, preferably 1% of hyaluronic acid or hyaluronic acid derivative.

Preferably, collagen type 1 or collagen type 3 is used in the composition. Of course, the use of a mixture of collagen type 1 and collagen type 3 in equal or different percentages is also within the scope of the invention. The collagen is then a mixed product of two collagens of a different type. The collagens used are prepared and purified for medical applications in a manner known to a person skilled in the art.

It is considered advantageous if a base material of the granular materials and/or the material forming the molded part is selected from the group consisting of aragonite, seashell, allogenic bone material, autogenic bone material, xenogenic bone material, FDBA (freeze-dried bone allocrafts), DFBDA (decalcified freeze-dried bone allocrafts), algae or algae extract, ceramics, calcium phosphate, in particular tricalcium phosphate or tetracalcium phosphate, calcium phosphate ceramics, bioglass, or mixtures thereof.

According to a preferred embodiment, the molded part and/or the granular material contain(s) allogenic material coated with collagen. An alternative provides that the molded part and/or the granular material consists completely of allogenic material.

In particular, it is conceivable to produce the molded part and the granular material from donor bone. Granular material produced from bone coming from bone banks, is likewise within the scope of the invention. The granular material thus obtained is coated with collagen and hyaluronic acid or is initially provided in a pure, uncoated form and mixed with collagen and hyaluronic acid upon preparation of the coating.

The invention also considers the use of FDBA (freeze-dried bone allocrafts), or of DFDBA (decalcified freeze-dried bone allocrafts) as advantageous. By forming the molded part and/or the granular material from a material taken from a genetically different individual of the same species, bone growth can optimally progress. The probability of inflammatory reactions is advantageously reduced.

The use of xenogenic materials for the production of the granular material and the subsequent bonding with collagen and/or hyaluronic acid has also proven to be advantageous.

Bovine, pig and horse bones, either in collagen-coated or pure, uncoated form, are particularly suitable for producing molded parts and granular materials suitable for humans. It is also possible and within the scope of the invention to provide the granular material from algae, in particular algae extracts, corals, or shells, and preferably coated with collagen or as a pure granular material without coating.

Seashells have been proven particularly suitable for producing the granular material, as they consist of a calcium/protein mixture, more exactly of aragonite, and can therefore be resorbed by the body particularly well.

In addition, it is also possible to produce the granular material from an autogenic material, i.e. a material provided by the patient, and to coat same with collagen. For this purpose, bone material is initially taken from the patient, same is processed to form granular material and prepared by means of coating with collagen and/or hyaluronic acid for use as a coating of a molded part, which is inserted or implanted in the patient within the course of further treatment. In this manner the probability of any occurrence of inflammatory reactions of the body of the patient is at its lowest.

Furthermore, it is possible to use alloplastic materials, such as calcium phosphates, ceramics, or bioglass for the production of the dimensionally stable granular material according to the invention, and to coat or cover same with collagen and/or hyaluronic acid and to use same as a coating composition.

Preferably, the base material of the granular material consists of:

aragonite in combination with 0 to 50%, in particular 15 to 35%, preferably 25% of bone material, in particular allogenic or autogenic bone material. The use of xenogenic bone material or one or more of the other materials listed above is likewise possible and within the scope of the invention. Combinations of various materials and the use thereof in combination with aragonite are also within the scope of the invention.

It is advantageous if the base material of the granular material is formed of only bone material, in particular of allogenic, autogenic, and/or xenogenic bone material.

Whereas the components of the composition in one preferred embodiment of the invention are not mixed until directly before the use thereof as a coating of the molded part, an alternative embodiment, being equally within the scope of the invention, provides that the base material of the granular material or the finished granular material has an enveloping layer of at least one collagen and/or hyaluronic acid or hyaluronic acid derivative. The granular material enveloped by collagen is provided separately in this application and is not mixed with hyaluronic acid until the insertion or application of the coating. It is likewise possible that the hyaluronic acid preparation is not used until the time of or during the insertion or application of the molded part coated with the granular material/collagen mixture on the bone as a rinsing solution or for the preparation of the insertion or application surface, and is mixed with the granular material/collagen mixture into the composition according to the invention.

Preferably, the granular material has a particle size of between 1 and 3 mm, in particular of between 1.1 and 2 mm, preferably of 1.5 mm. Said particle sizes or particle size ranges have proven to be optimal under resorption aspects. By means of the selection of the particle size being adjusted to the respective patient or to the respective intended use, the resorption duration and speed can be defined, and therefore the success of the treatment can be further improved. In addition to the particle size, the porosity of the granular material is another criterion to be considered. A high number of pores or pore bodies in the granular material or on the granular material surface can significantly enlarge the surface available for the epitaxial growth of vessels or osteoblasts and improve growth. The porosity of the granular material results from the material itself or can be adjusted in a defined range by means of appropriate pretreatment of the granular material or granular starting material or by means of an acid treatment or the like.

It is advantageous if a sealing material is provided between the molded part and the bone substrate in order to prevent epitaxial growth of vessels or the penetration of substances or microorganisms damaging bone growth into the granular material-filled or coated cavity provided by the molded part above the bone substrate. For this purpose, the sealing material is formed in particular from collagen, preferably collagen type 1 or type 3, or a mixture of collagen type 1 and collagen type 3, and/or hyaluronic acid or hyaluronic acid derivative.

A development of the invention, considered advantageous, provides that the composition contains at least one additional substance. Same is preferably selected from the group consisting of statin, vitamins, trace elements, antibiotics, or mixtures thereof. Whereas vitamins and trace elements serve to supply the newly formed cells, statins or statin preparations promote immune modulation and reduce the tendency to inflammation. Antibiotics serve to combat or avoid bacterial infections on or in the bone substrate. The invention does not remain limited to the previously mentioned substances, but includes all substances and substance mixtures known to the person skilled in the art, which are usable in the context of the present invention.

In this context, it is advantageous if the at least one additional substance has a proportion of the composition of 0.1 to 3%, in particular of 0.2 to 1.5%, preferably 0.25%.

Of equal inventive significance is a granular material, in particular for use in a composition for coating a molded part as described above and defined in the claims. The granular material is formed from a base material and has an enveloping layer of at least one collagen and hyaluronic acid or hyaluronic acid derivative that has been applied immediately after the production thereof or at the time of use. The base material of the granular material is preferably selected from the group consisting of aragonite, seashell, allogenic bone material, autogenic bone material, xenogenic bone material, FDBA (freeze-dried bone allocrafts), DFBDA (decalcified freeze-dried bone allocrafts), algae or algae extract, ceramics, calcium phosphate, in particular tricalcium phosphate or tetracalcium phosphate, calcium phosphate ceramics, bioglass or mixtures thereof, however, without limiting the invention thereto. The collagen enveloping the granular material or mixed with same is advantageously selected from the group consisting of collagen type 1 and type 3 or a mixture thereof.

An embodiment of the granular material according to the invention, which embodiment is considered advantageous, provides that the base material of the granular material consists of:

aragonite and 0 to 50%, in particular 15 to 35%, preferably 25% of bone material, in particular allogenic or autogenic bone material. In this context, it is considered advantageous if the bone material is in particular an allogenic, autogenic, and/or xenogenic bone material.

The base material of the granular material preferably has a particle size of between 1 and 3 mm, in particular of between 1.1 and 2 mm, preferably of 1.5 mm. Of course, the granular material can possess multiple fractions of various particle sizes. For example, the resorption speed and duration of the granular material in the body of the patient can be adjusted or adapted via the selection and distribution of the particle size(s).

A method for producing a granular material as defined above is also within the scope of the invention. The method comprises the following steps:

(i) sterilizing a starting material,

(ii) milling the starting material until reaching a milled product having a defined particle size

(iii) packaging the milled product.

It is advantageous if in step (i) an incubation of the starting material is carried out in sodium hypochloride. From said incubation, which is performed in particular for between 24 and 72 hours, preferably for 48 hours, still existing, optionally adhering organic material residues are dissolved from the starting material, and a sterile, contamination-free material is provided. The incubation is preferably followed by a drying step and/or additional incubation in an alcohol solution, in particular in ethanol or isopropanol. The substances used are permitted for the use in medicine and are of corresponding levels of purity. The treatment or processing of the starting material and of the finished granular material is carried out in the clean room or under clean room conditions using sterile devices.

An advantageous development of the method according to the invention further comprises, prior to packaging, the step (iia) of re-incubating the milled product in an alcohol solution, in particular in ethanol or isopropanol, and subsequent drying of the milled product.

In order to prevent any epitaxial growth of microorganisms during storage, a development of the method provides the step:

(iv) sterilizing the packaged milled product. In particular, sterilization is achieved by means of radiation using gamma radiation. Equally within the scope of the invention, however, are all other sterilization possibilities for the granular material or the packaged units that are known to a person skilled in the art and can be used in the context of the invention.

The invention also provides a use of a molded part as defined above and/or of a granular material as described above in medicine. The molded part, and/or the granular material are/is particularly suitable for use in plastic surgery or dentistry. Preferably, the use according to the invention is carried out in support of bone regeneration, in particular in the jawbone, wherein the molded part provides a cavity, that is filled with the granular material or coated therewith on the inner face thereof, as a space for bone regeneration.

### EXAMPLES

### Example 1

Toxicity test of the granular material according to the invention

The biological reactivity of mammal cell cultures (mouse fibroblast L929) in terms of exposure to the granular material according to the invention was examined. The granular material as described above was extracted in minimum essential medium (MEM), which had been supplemented by 10% fetal bovine serum (0.2 g/ml), for 24+/−2 hours at 37+/−1° C. Negative and positive controls were prepared in the same manner. The culture medium of the L929 cells, which had been grown in 96-well plates over 24+/−2 hours, was replaced in a total of six replicates by means of the extracts, and the cells were incubated for 24 to 26 hours at 37+/−1° C. The viability of the cells after exposure to the extracts was measured by measuring the absorption capacity thereof for a dye, neutral red. Said dye was added to the cells in order to be incorporated actively into viable cells. The number of viable cells correlates with the color intensity, which was determined by means of photometric measurements after extraction. The percentage of viable cells measured against the granular extract was 110%. The percentage of surviving cells exposed to negative and positive control substances was greater or smaller than 70%, by means of which the validity of the test was confirmed. Based on the criteria of the protocol and the ISO Standard 10993-5, 2009, the granular material has no cytotoxic potential.

### Example 2

Regeneration of osseous defects using a bone substitute based on aragonite (uncoated and coated with 5% of collagen) alone and in combination with 25% of autogenic bone.

The tested bone substitute serves for filling or bridging bone defects and lesions, which cannot be remedied by the body's own regeneration capability alone, and serves as a filler in reconstructive surgery, bone tumors or augmentations, such as before insertions of dental implants.

The bone substitute based on aragonite was tested in a so-called “critical size defect” model in terms of the osteogenic potency thereof, the variation over time of the osseous fusion, the variation over time of the material degradation, and in order to assess the mineralization content and the variation over time thereof. The experimental set-up provided for the use of 24 adult domestic pigs. In each of the animals, a total of eight osseous defects were produced in the Os frontale, having a diameter of 1 cm and a depth of 1 cm. The follow-up examination schedule was set for days 3, 7, 14, 21, 30, 56, 84, and 180 after surgery.

The test organisms were divided into a total of four experimental groups. The first group received a bone substitute consisting merely of aragonite, the second experimental group received a bone substitute consisting of aragonite and a 25% proportion of analogous bone, the third experimental group received a bone substitute made of aragonite, which was coated with collagen, the fourth group received a bone substitute which was formed from aragonite coated with collagen, and 25% of autogenic bone. The course of the osseous fusion was determined by means of microradiography. In microradiography, both material degradation and defect mineralization could be measured. Additionally, histological examinations were performed using toluidine blue staining. An immunohistochemical examination was likewise performed via collagen type 1 staining, osteocalcin staining, and Willebrand staining.

Results

After three days all experimental groups showed a comparable material degradation which was between 40 and 50%. The material degradation continuously declined, wherein again hardly any differences were detected between the materials used. The material degradation was fully completed after approximately 56 to 84 days. Contrary to the material degradation, a mineralization of the osseous defects could be determined.

Overall, the experiments were able to substantiate that
the degradation of the bone substitute was almost fully completed in all combinations after a standard time of 8 weeks. The degradation takes a nearly identical course in all aragonite combinations.
The combination of autogenic bone with an aragonite proportion has a slight advantage over the degradation of the bone substitute.
The bone regeneration achieves its maximum around the healing time of 8 weeks. The bone regeneration in the case of aragonite having collagen coating and in combination with autogenic bone is slightly increased as opposed to the other combinations.
Bone density is reduced during the time frame of 8 weeks to 12 weeks after surgery, which applies to all aragonite combinations, wherein thereafter an increase occurs up to the sixth month. The reasons are most probably remodeling processes within the bone (bone remodeling).

An increasing degradation of the bone substitute is observed in all experimental groups. Bone regeneration already begins around the 14^{th} day after surgery, and all modifications of the bone substitute based on aragonite show a complete regeneration of the osseous defects after 56 days.

Further advantages and purposeful embodiments are found in the claims, the description of the figures and the drawings which show:

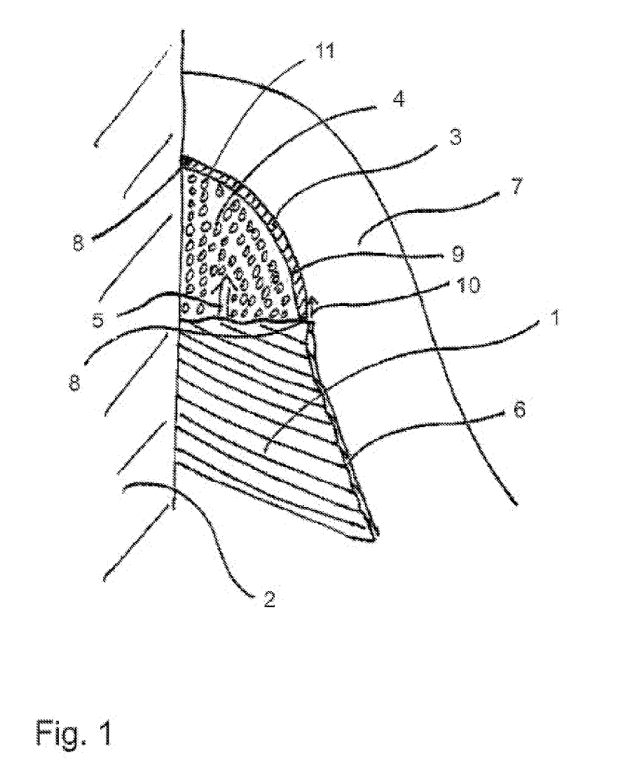

FIG. 1 a schematic illustration of a bone defect which was filled in with granular material,

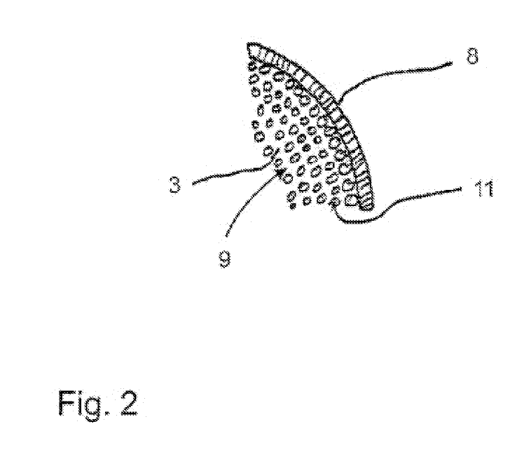

FIG. 2 a schematic sectional view of the coated molded part.

FIG. 1 shows a bone defect which is filled in with the granular material coated with the collagen or with liposomes or collagen liposomes, or with the granular material in pure form, i.e. without any coating. The illustration shows a jawbone 1 partially destroyed by periodontitis. If the jawbone 1 is not reconstructed, the tooth 2 held in the jawbone 1 can eventually fall out. For the purpose of bone regeneration, a granular material coated with collagen 8 or liposomes or collagen liposomes, or a granular material without any coating, but mixed with tetracycline powder and/or statin, is applied onto the jawbone 1. In FIG. 1, the molded part 3 is illustrated with granular material in a sectional view. The molded part 3 forms a cavity 4 above the jawbone 1, into which initially fibroblasts and then the osteoblasts of the jawbone 1 may grow in the direction of the arrow 5. In order to prevent cells of the periosteum 6 or cells of the gums 7 from penetrating the cavity 4, the molded part 3 and the granular material are sealed from the tooth 2 and from the jawbone 1 by means of collagen 8. In the shown embodiment, the molded part 3 forming a barrier consists of a seashell, which on the inner face 9 thereof has been adjusted to the special situation in the body of the patient by means of a coating composed of collagen, granular material and hyaluronic acid. Particular attention was paid to the resorption duration and suitability of the materials used, in particular of the granular material and the molded part. The longest required residence time of the molded part 3 and the granular material or the granular material residues on the jawbone 1 was defined in order to ensure satisfactory bone reconstruction.

In order to be able to insert the molded part 3 into the position illustrated, the upper gum flap 7 is initially folded open. Optionally, the surface of the jawbone 1 is roughened up in order to promote growth of the bone 1. Subsequently, the molded part 3 with the collagen/granular material/hyaluronic acid coating thereof is applied at the respective site, and, for example, bonded or fixed with pins at the jawbone 1 and/or tooth 2. Subsequently, the gum flap 7 is folded back to the position illustrated in FIG. 1 and fixed to the outer face of the molded part 3. The periosteum 6 grows on the outer face of the molded part 3 along the direction of the arrow 10 such that after some time the original jaw situation, including the entire jawbone 1, periosteum 6 and gums 7, is restored. A second surgery for removing the granular material 1 is not necessary after completed bone regeneration, since the granular material, molded part 3 and collagen 8, as well as hyaluronic acid are decomposed completely by the body.

FIG. 2 shows a molded part 3, the inner face 9 of which has been coated with a granular material. The granular material is a composition consisting of a granular material 11 which was produced on aragonite basis, has a coating with collagen 8 and to which hyaluronic acid has been added in addition. Due to the collagen 8 contained in the composition, same can be bonded to the inner face 9 of the molded part 3 and has a sufficiently good tendency to adhere at this location. The collagen component of the composition superficially coats the granular bodies and, in addition to the adherence to the inner face 9 of the molded part 3, promotes the ingrowth of the bone cells which remodel the jawbone 1 (cf. FIG. 1) piece by piece. Overall, the molded part 3 used provides a cavity, in which initially a blood clot is retained. Same serves as a base or framework for vascularization, which ultimately results in bone regeneration, if osteoblasts find a sufficient nutrient supply in order to form new bone material underneath the molded part 3 or in the cavity 4 formed thereby (cf. FIG. 1). Like the base material of the granular material 11, the molded part 3 is produced on aragonite basis and is completely resorbed by the body. Upon insertion of the molded part 3 in the jaw of a patient, the connecting points at the tooth 2 and at the jawbone 1 are additionally sealed by means of collagen 8. The entire insertion site of the molded part 3 is additionally rinsed with a hyaluronic acid solution. The coating composition additionally has bound hyaluronic acid, which is available during the ingrowth phase.

The claims submitted now together with the application and in the future are attempts of a formulation without prejudice for achieving further protection.

If, upon closer examination, in particular also of the relevant prior art, it should occur that one or another feature is favorable, but not critically essential for the goal of the invention, a formulation which does no longer comprise such a feature, particularly in the main claim, is already aimed at now, of course.

It should be further noted that the designs and variants of the invention described in the various embodiments and shown in the drawings can be interchangeably combined in any manner. For this purpose, individual or a plurality of features are interchangeable with one another in any manner. Said combinations of features are likewise disclosed.

The dependency references stated in the dependent claims indicate the further development of the subject matter of the main claim by means of the features of the respective dependent claim. However, these are not intended to be understood as waiving the achievement of an independent subject matter protection for the features of the back-referring dependent claims.

Features disclosed thus far only in the description may be claimed during the course of the procedure as being essential to the invention, for example, for the purpose of delimitation with respect to the prior art.

Features disclosed only in the description, or also individual features from claims comprising a plurality of features, may be included in the first claim at any time for the purpose of delimitation with respect to the prior art, even if such features were mentioned in combination with other features or achieve particularly favorable results in combination with other features.

1. Molded part for supporting bone regeneration, comprising:
a coating having a composition comprising;
(a) at least one collagen,
(b) a granular material, and
(c) hyaluronic acid or a hyaluronic acid derivative.

2. Molded part according to claim 16, wherein the coating is provided on a surface of the molded part facing the jawbone or the jawbone section, wherein an adherence of the granular material is provided by an adhesive.

3. Molded part according to claim 1 wherein the composition comprises;
1 to 1% of collagen,
99 to 80 of granular material,
0.01 to 2%, of hyaluronic acid or hyaluronic acid derivative,
and wherein the collagen is selected from the group consisting of collagen type 1 and type 3 or a mixture thereof.

4. Molded part according to claim 1 wherein at least one of a base material of the granular material and a material forming the molded part is selected from a group consisting essentially of aragonite, seashell, allogenic bone material, autogenic bone material, xenogenic bone material, FDBA (freeze-dried bone allocrafts), DFBDA (decalcified freeze-dried bone allocrafts), algae or algae extract, ceramics, and calcium phosphate.

5. Molded part according to claim 1 wherein a base material of the granular material comprises;
aragonite, and
0 to 50% of at least one of bone material, allogenic bone material and autogenic bone material.

6. Molded part according to claim 1 wherein a base material of the granular material has an enveloping layer of at least one collagen and hyaluronic acid or hyaluronic acid derivative, or mixtures thereof, and/or the granular material has a particle size of between 1 and 3 mm.

7. Molded part according to claim 1 wherein a sealing material is provided between the molded part and a jawbone or a bone substrate, and wherein the sealing material is formed from at least one of (a) collagen, (b) collagen type 1, and (c) a mixture of collagen type 1 and collagen type 3, combined with a hyaluronic acid or a hyaluronic acid derivative, and/or wherein the composition contains at least one further substance, wherein the at least one further substance is selected from the group consisting essentially of statin, vitamin, trace element, antibiotic or mixtures thereof, and wherein the at least one further substance has a proportion of 0.1 to 3% of the composition.

8. A granular material for use in a composition for coating the molded part as defined in claim 1, wherein a base material of the granular material has an enveloping layer of at least one collagen and hyaluronic acid or hyaluronic acid derivative, and wherein the base material of the granular material is selected from the group consisting essentially of aragonite, seashell, allogenic bone material, autogenic bone material, xenogenic bone material, FDBA (freeze-dried bone allocrafts), DFBDA (decalcified freeze-dried bone allocrafts), algae or algae extract, ceramics, at least one of calcium phosphate, tricalcium phosphate, tetracalcium phosphate, calcium phosphate ceramics, bioglass, and/or wherein the collagen is selected from the group consisting essentially of collagen type 1 and type 3 or a mixture thereof.

9. Granular material according to claim 8 wherein the base material of the granular material comprises;
aragonite, and
0 to 50% of at least one of bone material, allogenic bone material and autogenic bone material.

10. The granular material according to claim 8 wherein the base material of the granular material is formed from at least one of bone material, allogenic bone material, autogenic bone material and xenogenic bone material, and/or wherein the base material of the granular material has a particle size of 1 to 3 mm.

11. A method for producing a granular material according to claim 8, comprising the steps of:
(i) sterilizing a starting material,
(ii) milling the starting material until reaching a milled product having a defined particle size, and
(iii) packaging the milled product.

12. The method according to claim 11, wherein step (i) includes incubating of the starting material in sodium hypochloride for 24 to 72 hours.

13. The method according to claim 11, further comprising;
(ii)(a) incubating the milled product in at least one of alcohol, ethanol and isopropanol, and subsequently drying the milled product.

14. The method according to claim 11, further comprising;
(iv) sterilizing the packaged milled product by at least one of sterilizing energy, radiation and gamma radiation.

15. Use of the molded part according to claim 1 in at least one of medicine, plastic surgery and dentistry, for supporting bone regeneration, wherein the molded part provides a cavity filled with the granular material as a space for the bone regeneration.

16. Molded part according to claim 1 wherein the regeneration is regeneration of a jawbone or jawbone section in a mammal and, wherein the molded part is constructed and arranged for application to the jawbone or jawbone section.

17. Molded part according to claim 2 wherein the adhesive comprises at least one of a collagen a fibrin glue.

18. Molded part according to claim 2 wherein the coating and/or the molded part are fully resorbable by the mammal body.

19. Molded part according to claim 4 wherein the calcium phosphate comprises at least one of tricalcium phosphate or tetracalcium phosphate, calcium phosphate ceramics, bioglass, or mixtures thereof.

20. The method according to claim 12 further comprising drying and/or further incubating in at least one of alcohol, ethanol and isopropanol.
